(19) United States
(12) Patent Application Publication
(10) Pub. No.: US 2009/0291104 A1
(43) Pub. Date: Nov. 26, 2009

(54) NOVEL CONOTOXIN FRAMEWORK WITH A HELIX-LOOP-HELIX FOLD

(75) Inventor: Frank Mari, Boca Raton, FL (US)

Correspondence Address: Gregory A. Nelson;Novak Druce & Quigg LLP, 525 Okeechobee Blvd, Suite 1500, West Palm Beach, FL, 33401, US

(73) Assignee: Florida Atlantic University, Boca Raton, FL (US)

(21) Appl. No.: 11/917,127

(22) PCT Filed: Jun. 9, 2006
(86) PCT No.: PCT/US06/22461

§ 371 Date: Mar. 23, 2009

Related U.S. Application Data
(60) Provisional application No. 60/690,050, filed on Jun. 10, 2005.

(51) Int. Cl. A61K 9/00; C07K 7/50; A61K 38/12; A61K 35/12; C12N 5/10; C12N 15/87

(52) U.S. Cl. 424/400; 530/317; 514/11; 424/93.7; 435/325; 435/455

(57) ABSTRACT

A new family Here we report a new family of four-cystine/three-loop conotoxins (designated framework 14). Three peptides of this family (flf14a-c) were isolated from the venom of Conus floridanus floridensis and one (vil14a) from the venom of Conus villepinii, two worm-hunting Western Atlantic cone snail species. CD spectra and nanoNMR spectroscopy of these conotoxins directly isolated from the cone snails revealed a highly helical secondary structure for the four conotoxins. Sequence-specific nanoNMR analysis at room temperature revealed a well-defined helix-loop-helix tertiary structure that resembles that of the Cs α/α scorpion toxins κ-hefutoxin, κ-KTx1.3 and Om-toxins, which adopt a stable three-dimensional fold where the two α-helices are linked by the two disulfide bridges. One of these conotoxins (vil14a) has a Lys/Tyr (or Phe) diad, separated by approximately 6 Å, which is a conserved structural feature in K⁺ channel blockers.

### FIELD OF THE INVENTION

This invention relates generally to the fields of medicine and neuropharmacology. More particularly, the invention relates to a new class of conopeptide compounds useful for blocking ion channels.

### BACKGROUND OF THE INVENTION

After 40 years of research marine organisms have proven to be an excellent source of novel bioactive natural products. Increasingly, marine derived natural products advance into clinical trials and even more are currently in pre-clinical development.

Animals that utilize venom to capture prey, such as snakes, spiders, scorpions, sea anemones and cone snails, produce a plethora of cystine-stabilized peptidic scaffolds that target specifically ion channels and neuronal receptors as part of their neurochemical strategy for predation. Among them, cone snails, a genus of marine gastropods that can prey upon fish (piscivorous), mollusks (molluscivorous) and worms (vermivorous), contain in their venom an extraordinarily complex and diverse mixture of small neuroactive peptides (conopeptides) that specifically target ion channels and neuronal receptors. Conopeptides are important tools for investigating ion channel and receptor function and have great potential pharmacological applications. They can be classified into two major groups: (1) conotoxins, which contain two or more disulfide bonds and (2) those with only a single disulfide bridge or none at all, which are designated with trivial names such as contryphans, conantokins, contulakins, conorfamides, conophans and γ-hydroxyconophans. Conotoxins are grouped into various superfamilies (O, M, A, S, T, P, I), each with highly conserved signal sequences in their precursor proteins and a characteristic cystine arrangement in the mature peptides. Within the superfamilies, conotoxins are further classified into families according to their pharmacological targets, which include voltage-gated ion channels (Na⁺, K⁺ and Ca²⁺), ligand-gated ion channels (nAChR and 5-HT₃R), receptors (neurotensin type 1, α1 adrenergic, NMDA, Rfamide and vassopressin) and neurotransmitter transporters (NE).

Conopeptides inherently contain high degrees of modified amino acids (usually combinations of them), such as cystines, hydroxyproline, γ-carboxyglutamate, Br-Trp, D-Trp, D-Leu, D-Phe, D-IIe, D-Met, D-γ-hydroxyvaline, pyro-Glu, glycosylated Ser/Thr, and sulfated Tyr. These modifications confer conopeptides with unique stability and exquisite specificity towards neuronal targets, enabling cone snails to capture prey.

The precise composition of cone snail venom is species specific and it is the product of 55 million years of evolutionary refinement that has yielded a complex library of over 100,000 neuroactive conopeptides, as this genus comprises over 1000 species distributed in the tropical and sub-tropical areas of Atlantic, Indian and Pacific oceans. Only a small fraction of this immense conopeptide library has been analyzed to date (<0.2%) and many novel conopeptide frameworks are yet to be discovered. Most conopeptides that have been isolated and characterized are from Conus species found in the Indo-Pacific region.

There is a need in the art for the development of new drugs which are useful for treating pain, cancer, neurological diseases and the like.

### SUMMARY

The invention relates to the isolation, synthesis and therapeutic use of compounds and related compositions based on a new class of Conus conopeptides of a novel four-cystine/three-loop conotoxin framework (framework 13) from Conus villepinii (vil13a) and Conus floridanus floridensis (flf 13a-c). These new conotoxins are 27-residue polypeptide chains with a 1-4/2-3 cystine pairing. NanoNMR analysis revealed a well-defined helix-loop-helix three-dimensional fold where the two α-helices are linked by the two disulfide bridges. This tertiary structure resembles that of the Cs α/α toxins recently found in scorpion venom that targets the potassium channels.

In a preferred embodiment, an isolated conopeptide, comprises a framework characterized by a 4-Cys/3-loop sequence with a conserved loop spacing ¹Cys-X₃₋²Cys-X₁₁-³Cys-X₃-⁴Cys and Cys pairing 1-4/2-3.

In another preferred embodiment, the conopeptide comprises a Lys/Tyr diad.

In another preferred embodiment, the peptide is at least one of SEQ ID NO's: 1-4 and 14-20.

In another preferred embodiment, a composition comprises an isolated conopeptide, wherein the isolated conopeptide comprises a framework characterized by a 4-Cys/3-loop sequence with a conserved loop spacing ¹Cys-X₃-²Cys-X₁₁-³Cys-X₃-⁴Cys and Cys pairing 1-4/2-3 in a pharmaceutically acceptable carrier. In one aspect, the conopeptide comprises a comprises a Lys/Tyr (or Phe) diad. The composition comprises at least one of SEQ ID NO's: 1-4 and 14-20.

In another preferred embodiment, the isolated conopeptide comprises at least one of SEQ ID NO's.: 1-4 and 14-20.

In another preferred embodiment, a composition comprises at least one of SEQ ID NO's.: 1-4 and 14-20 in a pharmaceutical carrier.

In another preferred embodiment, a method of inducing analgesia in a mammal comprises administering a therapeutically effective amount of a conopeptide identified by an one of SEQ ID NO's.: 1-4 and 14-20. The administration comprises using a delivery means selected from the group consisting of a pump, microencapsulation, a continuous release polymer implant, microencapsulation, naked or unencapsulated cell grafts, injection and oral administration. Preferably, the amount of conopeptide administered is between about 0.001 mg/kg to about 250 mg/kg.

In another preferred embodiment, a pharmaceutical composition comprises a therapeutically effective amount of a conopeptide identified by at least one of SEQ ID NO's.: 1-4 and 14-20 or a pharmaceutically acceptable salt or solvate thereof and a pharmaceutically acceptable carrier. The composition can further comprise one or more drugs useful in the treatment of pain.

In another preferred embodiment, a method of treating cancer comprises administering a therapeutically effective amount of a conopeptide identified by an one of SEQ ID NO's.: 1-4 and 14-20 to a patient in need thereof; killing the cancer cells and, treating cancer.

In another preferred embodiment, an isolated cell comprises a vector expressing a conopeptide, comprising a framework characterized by a 4-Cys/3-loop sequence with a conserved loop spacing ¹Cys-X₃-²Cys-X₁₁-³Cys-X₃-⁴Cys and Cys pairing 1-4/2-3. The expressed conopeptide comprises a Lys/Tyr (or Phe) diad. Preferably, the expressed peptide is at least one of SEQ ID NO's: 1-4 and 14-20.

In another preferred embodiment, a method of blocking potassium channels in a cell comprises transforming a cell with a vector expressing a conopeptide, comprising a framework characterized by a 4-Cys/3-loop sequence with a conserved loop spacing ¹Cys-X₃-²Cys-X₁₁-³Cys-X₃-⁴Cys and Cys pairing 1-4/2-3; and, culturing said cell, wherein, expression of the conopeptide blocks the potassium channel. The expressed conopeptide can comprise a Lys/Tyr (or Phe) diad. Preferably, the expressed conopeptide is at least one of SEQ ID NO's: 1-4 and 14-20.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIGS. 1A and 1B are scans of a photograph showing the shells of C. villepinii (FIG. 1A) and C. floridanus floridensis (FIG. 1B).

FIGS. 2A and 2B are elution profiles showing the purification of peptides by HPLC-Superdex 30. The column was eluted with 0.1 M NH₄HCO₃ at a flow of 1.5 ml/min. FIG. 2A shows the fractionation of the venom of C. floridanus floridensis. Peptides flf14a, flf14b and flf14c were purified from the peaks identified by the arrows. FIG. 2B shows the separation of the venom of C. villepinii. Peptide vil14a was purified from the peak marked by an arrow.

FIGS. 3A-3D are elution profiles showing the purification of peptides: flf14a (FIG. 3A), flf14b (FIG. 3B), flf14c (FIG. 3C) and vil14a (FIG. 3D). Fractions marked with the arrows on FIGS. 2A and 2B were applied to a Vydac C18 semipreparative column and eluted with a linear gradient of 1% buffer B increase/min for 100 min at a flow of 3.5 ml/min. The peaks highlighted by the arrows were further purified on an analytical column Vydac C18 using the same gradient for 100 min at 1 ml/min (embedded figures). For both semipreparative and analytical RP-HPLC, the buffers were 0.1% TFA (buffer A) and 0.1% TFA in 60% acetonitrile (buffer B).

FIG. 4 shows a schematic representation of peptides digested with cyanogen bromide (CNBr) and chymotrypsin (Chym). Peptide fragments and molecular weight depend on the disulfide pattern. X₁ and X₂ are either Phe, Trp, or Tyr.

FIGS. 5A-5C are spectrographs showing MALDI TOF mass spectra (reflector mode) of the peptides flf14a (FIG. 5A), flf14b (FIG. 5B) and vil14a (FIG. 5C) after treatment with CNBr/chymotrypsin. Arrows highlight some molecular weights and the corresponding fragments are shown. Monoisotopic molecular weights were compared with the calculated molecular weights.

FIG. 6 is a spectrograph showing the CD spectra of native peptides flf14a, flf14b, flf14c and vil14a in H₂O at 25° C. The table below shows the relative α-helix content for each peptide.

FIGS. 7A-7C show the 2D-NOESY spectra of flf14b (FIG. 7A) and vil14a (FIG. 7B) recorded at 25° C. using a 1.7 mm NMR tube in 3 mm gHCN probe. Water suppression was achieved using Watergate. Regions containing the NN(i,i+1) and αN(i,i+1) correlations are outlined with rectangular boxes. FIG. 7C shows the NN(i,i+1) sequence specific assignments of vil14a.

FIG. 8A shows the sequential NOEs of vil14a (sequence shown at the top). The relative strengths of the sequential NOEs are indicated by thickness of the bars. Two α-helical segments were found between residues 6-12 and 18-26. FIG. 8B is graph showing the proton chemical shift index (CSI) which indicates the presence of two helical segments in the peptide separate by a loop. FIG. 8C is a schematic representation of a model of the lowest energy structure depicting the backbone ribbon fold and disulfide bridges of vil14a. Lys23 is shown along with two proximal Tyr residues (8 and 27).

FIGS. 9A-9C are graphs showing the effect of Vil14a on Kv1.3. Cells were clamped at Vhold=−90 mV and 240 ms depolarizaing steps from Vtest=−70 to 40 mV every 5 s, in the absence and presence of 10 μM Vil14a. FIG. 9B is a graph showing % of block vs voltage. FIG. 9C is a graph showing steady-state current-voltage relationship (n=3), control (open circles), with 10 μM Vil14a (filled circles), after washing the toxin (filled triangles dashed lines) V50 control=−1.5 mV, toxin 10 μM 19 mV, wash=−4.4 mV.

FIGS. 10A-10C are graphs showing the effect of expression of Vil 14a in HEK293 cells. FIG. 10A shows current records of Kv1.3 at a test potential of +40 mV before and after adding 100 μM Vil14a (holding potential −90 mV). FIG. 10B shows the steady-state activation curve in control and 100 μM Vil14a obtained after fitting with a Boltzmann function (I═I_{c}/(1+exp(−(x−V₅₀)/s)) V50=−9 mV (control), V50 toxin=+9 mV. FIG. 10C shows a dose-response curve as a plot of the percentage of unblocked currents as a function of increasing toxin concentration. Each point is the mean±SEM for 3 independent experiments. The solid line through the data is a fit of % of unblocked currents measured at +40 mV with a K_{d}=15.6 μM. dose-response for a bimolecular reaction. I_{C} is the control current.

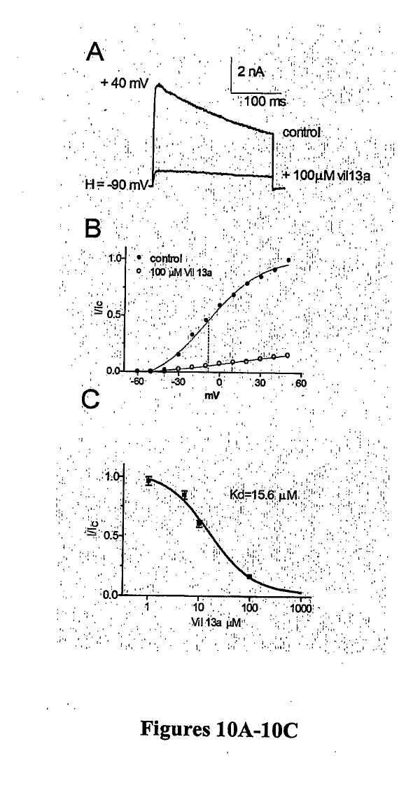

### DETAILED DESCRIPTION

The invention provides for a new family of four-cystine/three-loop conotoxins (designated framework 14). Three peptides of this family (flf14a-c) were isolated from the venom of Conus floridanus floridensis and one (vil14a) from the venom of Conus villepinii, two worm-hunting Western Atlantic cone snail species. The isolated peptides are effective ion channel blockers.

### DEFINITIONS

The present section provides definitions of the terms used in the present invention in order to facilitate a better understanding of the invention.

As used herein, the singular forms “a”, “an” and “the” include plural referents unless the context clearly dictates otherwise.

The term “subject,” or “patient” as used herein, means a human or non-human animal, including but not limited to mammals such as a dog, cat, horse, cow, pig, sheep, goat, chicken, primate, rat, and mouse.

An “expression vector” is a vector capable of expressing a DNA (or cDNA) molecule cloned into the vector and, in certain cases, producing a polypeptide or protein. Appropriate transcriptional and/or translational control sequences are included in the vector to allow it to be expressed in a cell. Expression of the cloned sequences occurs when the expression vector is introduced into an appropriate host cell. If a eukaryotic expression vector is employed, then the appropriate host cell would be any eukaryotic cell capable of expressing the cloned sequences.

As used herein, the term “administering a molecule to a cell” (e.g., an expression vector, nucleic acid, a angiogenic factor, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

A cell has been “transformed”, “transduced”, or “transfected” by exogenous or heterologous nucleic acids when such nucleic acids have been introduced inside the cell. Transforming DNA may or may not be integrated (covalently linked) with chromosomal DNA making up the genome of the cell. In prokaryotes, yeast, and mammalian cells for example, the transforming DNA may be maintained on an episomal element, such as a plasmid. In a eukaryotic cell, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations (e.g., at least about 10).

A “vector” is a composition which can transduce, transfect, transform or infect a cell, thereby causing the cell to express nucleic acids and/or proteins other than those native to the cell, or in a manner not native to the cell. A cell is “transduced” by a nucleic acid when the nucleic acid is translocated into the cell from the extracellular environment. Any method of transferring a nucleic acid into the cell may be used; the term, unless otherwise indicated, does not imply any particular method of delivering a nucleic acid into a cell. A cell is “transformed” by a nucleic acid when the nucleic acid is transduced into the cell and stably replicated. A vector includes a nucleic acid (ordinarily RNA or DNA) to be expressed by the cell. A vector optionally includes materials to aid in achieving entry of the nucleic acid into the cell, such as a viral particle, liposome, protein coating or the like. A “cell transduction vector” is a vector which encodes a nucleic acid capable of stable replication and expression in a cell once the nucleic acid is transduced into the cell.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference.

### Conopeptides and Uses Thereof

The primary structure for these peptides was determined using Edman degradation sequencing and their cystine pairing was assessed by limited hydrolysis with a combination of CNBr and chymotrypsin under non-reducing, non-alkylating conditions in combination with MALDI-TOF MS analysis of the resulting peptidic fragments. CD spectra and nanoNMR spectroscopy of these conotoxins directly isolated from the cone snails revealed a highly helical secondary structure for the four conotoxins. Sequence-specific nanoNMR analysis at room temperature revealed a well-defined helix-loop-helix tertiary structure that resembles that of the Cs α/α scorpion toxins κ-hefutoxin, κ-KTx1.3 and Om-toxins, which adopt a stable three-dimensional fold where the two α-helices are linked by the two disulfide bridges. One of these conotoxins (vil14a) has a Lys/Tyr diad, separated by approximately 6 Å, which is a conserved structural feature in K⁺ channel blockers. The presence of this framework in scorpions and in cone snails indicates a common molecular imprint in the venom of apparently unrelated predatory animals and suggests a common ancestral genetic origin.

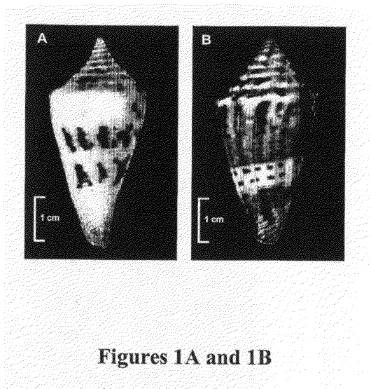

As part of our efforts towards the analysis and characterization of conopeptides isolated from cone snail species from the Americas, we proceeded with the isolation and structural analysis of conopeptides from Conus floridanus floridenses and Conus villepinii, which are widespread worm-hunting cone snail species of the Western Atlantic Ocean (FIGS. 1A and 1B). C. villepinii is a deep-water species (>100 m) and its habitat ranges from the Florida coastline and the Gulf of Mexico to Brazil. C. floridanus is also a Western Atlantic species whose range varies from Florida to the Yucatan Peninsula; however, unlike C. villepinii, C floridanus inhabits shallow sandy areas. Four variants exist of C. floridanus: C. floridanus floridensis, C. floridanus buryae, C.floridanus patglicksteinae and C. floridanus yucatanensis; these variants of the C. floridanus complex are biogeographical subspecies that show differences in their venom composition. To date, no peptides have been isolated from the venom of these Western Atlantic Conus species.

The peptides of the invention are of interest because potassium channels are a ubiquitous group of ion channels that are important in controlling excitability and modulating secretory processes. They have a number of roles including neuronal integration, volume regulation, maintenance of the resting membrane potential and an important role in determining the frequency and duration of action potentials. The changes in cell excitability that follow modulation of potassium channels give rise to a broad number of potential therapeutic uses of such modulators. One example is the potassium channel blocker, dofetilide, which is an effective antiarrhythmic.

In a preferred embodiment, a composition comprising at least one of conopeptides, SEQ ID NO's: 1-4 and 14-20 are used to treat or prevent a variety of cardiac pathological conditions that either singly or together with one or more additional compounds are able to selectively inhibit cardiac ionic currents.

Early repolarising currents correspond to those cardiac ionic currents which activate rapidly after depolarization of membrane voltage and which effect repolarisation of the cell. Many of these currents are potassium currents and may include, but are not limited to, the transient outward current Iₜₒ₁, such as Kv4.2 and Kv4.3), and the ultrarapid delayed rectifier current (I_{Kur}) such as Kv1.5, Kv1.4 and Kv2.1). The ultrarapid delayed rectifier current (I_{Kur}) has also been described as I_{SUS}. A second calcium dependent transient outward current (1ₜₒ₂) has also been described.

The cardiac pathological conditions that may be treated and/or prevented by the present invention may include, but are not limited to, arrhythmias such as the various types of atrial and ventricular arrhythmias.

In another embodiment, the present invention provides ion channel modulating compounds that can be used to selectively inhibit cardiac early repolarising currents and cardiac sodium currents under conditions where an “arrhythmogenic substrate” is present in the heart. An “arrhythmogenic substrate” is characterized by a reduction in cardiac action potential duration and/or changes in action potential morphology, premature action potentials, high heart rates and may also include increased variability in the time between action potentials and an increase in cardiac milieu acidity due to ischemia or inflammation. Changes such as these are observed during conditions of myocardial ischemia or inflammation and those conditions that precede the onset of arrhythmias such as atrial fibrillation.

In a preferred embodiment, a method of blocking ion channels in a cell comprises transforming a cell with a vector expressing a conopeptide, comprising a framework characterized by a 4-Cys/3-loop sequence with a conserved loop spacing ¹Cys-X₃-²Cys-X₁₁-³Cys-X₃-⁴Cys and Cys pairing 1-4/2-3; and, culturing said cell, wherein, expression of the conopeptide blocks the potassium channel. The expressed conopeptide can comprise a Lys/Tyr (or Phe) diad. Preferably, the expressed conopeptide is at least one of SEQ ID NO's: 1-4 and 14-20.

In other embodiments, the present invention provides pharmaceutical compositions that contain at least one peptide of SEQ ID NO's: 1-4 and 14-20 in an amount effective to treat a disease or condition in a warm-blooded animal suffering from or having the disease or condition, and/or prevent a disease or condition in a warm-blooded animal that would otherwise occur, and further contains at least one pharmaceutically acceptable carrier, diluent or excipient. The invention further provides for methods of treating a disease or condition in a warm-blooded animal suffering from or having the disease or condition, and/or preventing a disease or condition from arising in a warm-blooded animal, wherein a therapeutically effective amount of at least one peptide of SEQ ID NO's: 1-4 and 14-20, or a composition containing at least one peptide of SEQ ID NO's: 1-4 and 14-20 is administered to a warm-blooded animal in need thereof. The diseases and conditions to which the compounds, compositions and methods of the present invention have applicability are as follows: arrhythmia, diseases of the central nervous system, convulsion, epileptic spasms, depression, anxiety, schizophrenia, Parkinson's disease, respiratory disorders, cystic fibrosis, asthma, cough, inflammation, arthritis, allergies, gastrointestinal disorders, urinary incontinence, irritable bowel syndrome, cardiovascular diseases, cerebral or myocardial ischemias, hypertension, long-QT syndrome, stroke, migraine, ophthalmic diseases, diabetes mellitus, myopathies, Becker's myotonia, myasthenia gravis, paramyotonia congentia; malignant hyperthermia, hyperkalemic periodic paralysis, Thomsen's myotonia, autoimmune disorders, graft rejection in organ transplantation or bone marrow transplantation, heart failure, hypotension, Alzheimer's disease or other metal disorder, and alopecia.

In another embodiment, the present invention provides a pharmaceutical composition containing an amount of at least one peptide of SEQ ID NO's: 1-4 and 14-20 effective to produce local analgesia or anesthesia in a warm-blooded animal in need thereof, and a pharmaceutically acceptable carrier, diluent, or excipient. The invention further provides a method for producing, local analgesia or anesthesia in a warm-blooded animal which includes administering to a warm-blooded animal in need thereof an effective amount of at least one peptide of SEQ ID NO's: 1-4 and 14-20 or a pharmaceutical composition containing at least one peptide of SEQ ID NO's: 1-4 and 14-20. These compositions and methods may be used to relieve or forestall the sensation of pain in a warm-blooded animal.

In another embodiment, the present invention provides a pharmaceutical composition containing an amount of at least one peptide of SEQ ID NO's: 1-4 and 14-20 effective to enhance the libido in a warm-blooded animal in need thereof, and a pharmaceutically acceptable carrier, diluent, or excipient. The invention further provides a method for enhancing libido in a warm-blooded animal which includes administering to a warm-blooded animal in need thereof an effective amount of at least one peptide of SEQ ID NO's: 1-4 and 14-20 or a pharmaceutical composition containing at least one peptide of SEQ ID NO's: 1-4 and 14-20. These compositions and methods may be used, for example, to treat a sexual dysfunction, e.g., impotence in males, and/or to enhance the sexual desire of a patient without a sexual dysfunction. As another example, the therapeutically effective amount may be administered to a bull (or other breeding stock), to promote increased semen ejaculation, where the ejaculated semen is collected and stored for use as it is needed to impregnate female cows in promotion of a breeding program.

In another embodiment, the present invention provides at least one peptide of SEQ ID NO's: 1-4 and 14-20 or composition containing at least one peptide of SEQ ID NO's: 1-4 and 14-20, for use in methods for either modulating ion channel activity in a warm-blooded animal or for modulating ion channel activity in vitro.

Methods of treating and/or preventing arrhythmia, one or more ion channel modulating peptides, either singly or together with one or more additional peptides, are used to inhibit cardiac ionic currents. Methods for in vitro assessment of inhibition activity of ion channel modulating compounds are well known in the art and are briefly described in the Examples which follow. The present invention provides that the above-described ion channel modulating peptides and/or composition(s) containing same may be used in a method for treating or preventing arrhythmia in a warm-blooded animal; and/or may be used in a method for modulating ion channel activity in a warm-blooded animal; and/or may be used in a method for modulating ion channel activity in vitro. The invention also provides for the use of an ion channel modulating compound in a manufacture of a medicament.

The invention further provides a pharmaceutical composition comprising (a) an amount of an ion modulating compound as described above effective to treat or prevent atrial arrhythmia in a warm-blooded animal in need of the treatment or prevention, and (b) a pharmaceutically acceptable carrier, diluent, or excipient. According to the present invention, this composition may be used in a method for treating or preventing atrial arrhythmia in a warm-blooded animal, where the method comprises administering to a warm-blooded animal in need thereof a therapeutically effective amount of one of the above-described ion channel modulating peptides or a composition containing same.

The invention further provides a pharmaceutical composition comprising (a) an amount of an ion channel modulating peptides effective to treat or prevent ventricular arrhythmia in a warm-blooded animal in need of the treatment or prevention, and (b) a pharmaceutically acceptable carrier, diluent, or excipient. This composition may be used in a method for treating or preventing ventricular arrhythmia in a warm-blooded animal, where the method comprises administering to a warm-blooded animal in need thereof a therapeutically effective amount of one of the above-described ion channel modulating peptides or a composition containing same.

### Animal Subjects

Because control of ion flux in cells is central to a very large number of physiological processes in animals and man, the invention is believed to be compatible with any animal subject. A non-exhaustive list of examples of such animals includes mammals such as mice, rats, rabbits, goats, sheep, pigs, horses, cattle, dogs, cats, and primates such as monkeys, apes, and human beings. Those animal subjects that have a disease or condition that relates to modulation of calcium levels within a cell are preferred for use in the invention, as these animals may have the symptoms of their disease reduced or even reversed. In particular, human patients suffering from cardiac disorders such as arrhythmia, neurologic disorders such as Alzheimer's disease, immune system dysfunction, and cancer can particularly benefit.

### Administration of Compositions

The compositions of the invention may be administered to animals including humans in any suitable formulation. For example, the compositions may be formulated in pharmaceutically acceptable carriers or diluents such as physiological saline or a buffered salt solution. Suitable carriers and diluents can be selected on the basis of mode and route of administration and standard pharmaceutical practice. A description of other exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. Such administration may be oral or parenteral (for example, by intravenous, subcutaneous, intramuscular, or intraperitoneal introduction). The compositions may also be administered directly to the target site by, for example, surgical delivery to an internal or external target site, or by catheter to a site accessible by a blood vessel. Other methods of delivery, for example, liposomal delivery or diffusion from a device impregnated with the composition, are known in the art. The compositions may be administered in a single bolus, multiple injections, or by continuous infusion (for example, intravenously or by peritoneal dialysis). For parenteral administration, the compositions are preferably formulated in a sterilized pyrogen-free form.

Compositions of the invention can also be administered in vitro to a cell (for example, in in vitro assays to modulate calcium levels within the cells, or to target particular cell surface receptors capable of selectively binding these peptides).

The compositions comprising at least one of SEQ ID NO's.: 1-4 and 14-20, as the active ingredient, and pharmaceutical acceptable salts. Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion. For example, administration of the active agent according to this invention may be achieved using any suitable delivery means, including: (a) pump (see, e.g., Annals of Pharmacotherapy, 27:912 (1993); Cancer, 41:1270 (1993); Cancer Research, 44:1698 (1984)); (b) microencapsulation (see, e.g., U.S. Pat. Nos. 4,352,883; 4,353,888; and 5,084,350); (c) continuous release polymer implants (see, e.g., U.S. Pat. No. 4,883,666); (d) macroencapsulation (see, e.g., U.S. Pat. Nos. 5,284,761, 5,158,881, 4,976,859 and 4,968,733 and published PCT patent applications WO92/19195, WO 95/05452); (e) naked or unencapsulated cell grafts to the CNS (see, e.g., U.S. Pat. Nos. 5,082,670 and 5,618,531); (f) injection, either subcutaneously, intravenously, intra-arterially, intramuscularly, or to other suitable site; or (g) oral administration, in capsule, liquid, tablet, pill, or prolonged release formulation.

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cells, by the use of targeting systems such as antibodies or cell-specific ligands. Targeting may be desirable for a variety of reasons, e.g. if the agent is unacceptably toxic, if it would otherwise require too high a dosage, or if it would not otherwise be able to enter target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Pat. No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines, other conopeptides and other therapeutic agents useful in all of the major areas of clinical medicine. When used with other pharmaceutically active compounds, the conopeptides of the present invention may be delivered in the form of drug cocktails. A cocktail is a mixture of at least one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

As disclosed herein, the compounds and compositions of the present invention are useful in treating pain. As such, they may also be useful in treating inflammatory pain. Accordingly, the compounds and compositions of the present invention may also be utilized to treat numerous inflammatory disease states and disorders other than pain. For example, the compositions and compounds may be useful for treating disorders or diseases including but not limited to: Alzheimer's disease, multiple sclerosis, attenuation of morphine withdrawal, cardiovascular changes, edema, such as edema caused by thermal injury, chronic inflammatory diseases such as rheumatoid arthritis, asthma/bronchial hyperreactivity and other respiratory diseases including allergic rhinitis, inflammatory diseases of the gut including ulcerative colitis and Crohn's disease, ocular injury and ocular inflammatory diseases, proliferative vitreoretinopathy, irritable bowel syndrome and disorders of bladder function including cystitis and bladder detrusor hyperreflexia, demyelinating diseases such as multiple sclerosis and amyotrophic lateral sclerosis, asthmatic disease, small cell carcinomas, in particular small cell lung cancer, depression, dysthymic disorders, chronic obstructive airways disease, hypersensitivity disorders such as poison ivy, vasospastic diseases such as angina and Reynauld's disease, fibrosing and collagen diseases such as scleroderma and eosinophilic fascioliasis, reflex sympathetic dystrophy such as shoulder/hand syndrome, addiction disorders such as alcoholism, stress related somatic disorders, neuropathy, neuralgia, disorder related to immune enhancement or suppression such as systemic lupus erythmatosis conjunctivitis, vernal conjunctivitis, contact dermatitis, atopic dermatitis, urticaria, and other eczematoid dermatitis and emesis; central nervous system disorders such as anxiety, depression, psychosis and schizophrenia; neurodegenerative disorders such as AIDS related dementia, senile dementia of the Alzheimer type, Alzheimer's disease and Down's syndrome; demyelinating diseases such as multiple sclerosis (MS) and amyotrophic lateral sclerosis (ALS; Lou Gehrig's disease) and other neuropathological disorders such as peripheal neuropathy, inflammatory diseases such as inflammatory bowel disease, irritable bowel syndrome, psoriasis, fibrositis, ocular inflammation, osteoarthritis and rheumatoid arthritis; allergies such as eczema and rhinitis; hypersensitivity disorders such as poison ivy; ophthalmic diseases such as conjunctivitis, vernal conjunctivitis, dry eye syndrome, and the like; cutaneous diseases such as contact dermatitis, atopic dermatitis, urticaria, and other eczematoid dermatitis; oedema, such as oedema caused by thermal injury; addiction disorders such as alcoholism; stress related somatic disorders; reflex sympathetic dystrophy such as shoulder/hand syndrome; dysthymic disorders; neuropathy, such as diabetic or peripheral neuropathy and chemotherapy-induced nemopathy; postherpetic and other neuralgias; asthma; osteoarthritis; rheumatoid arthritis; migraine reperfusion injury to an ischemic organ, e.g., reperfusion injury to the ischemic myocardium, myocardial infarction, inflammatory bowel disease, rheumatoid arthritis, osteoarthritis, hypertension, psoriasis, organ transplant rejections, organ preservation, impotence, radiation-induced injury, asthma, atherosclerosis, thrombosis, platelet aggregation, metastasis, influenza, stroke, burns, trauma, acute pancreatitis, pyelonephritis, hepatitis, autoimmune diseases, insulin-dependent diabetes mellitus, disseminated intravascular coagulation, fatty embolism, adult and infantile respiratory diseases, carcinogenesis and hemorrhages among many others.

### Effective Doses

An effective amount is an amount which is capable of producing a desirable result in a treated animal or cell (for example, reduced calcium flux in the cells of the animal or in a cell in culture). As is well known in the medical and veterinary arts, dosage for any one animal depends on many factors, including the particular animal's size, body surface area, age, the particular composition to be administered, time and route of administration, general health, and other drugs being administered concurrently. It is expected that an appropriate dosage for parenteral or oral administration of compositions of the invention would be in the range of about 1 μg to 100 mg/kg of body weight in humans. An effective amount for use with a cell in culture will also vary, but can be readily determined empirically (for example, by adding varying concentrations to the cell and selecting the concentration that best produces the desired result). It is expected that an appropriate concentration would be in the range of about 0.0001-100 mM. More specific dosages can be determined, for example, using a cultured neuronal cell assay as described below.

The active agent is preferably administered in an therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington's Pharmaceutical Sciences.

Dosage may be adjusted appropriately to achieve desired drug levels, locally or systemically. Typically the conopeptides of the present invention exhibit their effect at a dosage range from about 0.001 mg/kg to about 250 mg/kg, preferably from about 0.05 mg/kg to about 100 mg/kg of the active ingredient, more preferably from about 0.1 mg/kg to about 75 mg/kg, and most preferably from about 1.0 mg/kg to about 50 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example 24 hours or multiple doses per day are contemplated to achieve appropriate systemic levels of compounds.

Toxicity and efficacy of the compositions of the invention can be determined by standard pharmaceutical procedures, using cells in culture and/or experimental animals to determine the LD₅₀ (the dose lethal to 50% of the population) and the ED₅₀ (the dose that effects the desired result in 50% of the population). Compositions that exhibit a large LD₅₀/ED₅₀ ratio are preferred. Although less toxic compositions are generally preferred, more toxic compositions may sometimes be used in in vivo applications if appropriate steps are taken to minimize the toxic side effects.

Data obtained from cell culture and animal studies can be used in estimating an appropriate dose range for use in humans. A preferred dosage range is one that results in circulating concentrations of the composition that cause little or no toxicity. The dosage may vary within this range depending on the form of the composition employed and the method of administration.

Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

### Materials and Methods

Abbreviations: The abbreviations used are: DSV, deep submersible vehicle; TFA, trifluoroacetic acid; SE, size exclusion; HPLC, high performance liquid chromatography; RP, reversed phase; DTT, dithiothreitol; LAM, iodoacetamide; MALDI-TOF, matrix assisted laser desorption ionization time-of-flight; CNBr, cyanogen bromide; PFG, pulse field gradient; RF, radio frequency; TSP, 3-(Trimethylsilyl)-Propionic acid-D4, sodium salt; WET, water suppression enhanced through Ti effects; NOE, nuclear Overhauser effect; NOESY, nuclear Overhauser effect spectroscopy; TOCSY, total correlated spectroscopy; BLAST, basic local alignment search tool; Cs β/β, Cystine-stabilized P/P; Cs α/α, Cystine-stabilized α/α.

Specimen collection. Specimens of C. villepinii (30-80 mm) were collected off the Florida Keys Narathon Key), USA, using a Capetown dredge deployed from the oceanic research vessels R/V Suncoaster and R/V Bellows at depths ranging from 100-200 m. Additional snails were collected using the Johnson-Sea-Link deep submersible vehicle operated from the R/V Seward Johnson and working at a 200 m depth at the same location indicated above. Cone snails were collected using a suction device attached to a robotic arm of the DSV. Specimens of C. floridanus floridensis (20-45 mm) were collected in the southwest coast of Florida, USA, in sandy areas at low tides. All snails were kept in aquaria prior to transportation to the lab, where there were dissected and immediately frozen at −80° C.

### Crude Venom Extraction.

Venom ducts dissected from either 63 specimens of C. villepinii or 47 specimens of C. floridanus floridensis were homogenized in 0.1% TFA at 4° C. Whole extracts were centrifuged at 10,000×g for 20 min, at 4° C., and the resulting pellets were washed three times with 0.1% TFA and re-centrifuged under identical conditions. The supernatants containing the soluble peptides were pooled, lyophilized, and stored at −80° C. until further use.

### Peptide Purification.

Crude venom were initially fractionated by SE-HPLC on a Pharmacia Superdex-30 column (2.5×100 cm) equilibrated and eluted with 0.1 M NH₄HCO₃ using a flow rate of 1.5 ml/min. Chromatographic fractions were monitored at λ values of 220, 250, and 280 nm. Additional purification of peptide-containing peaks was achieved by RP-HPLC on a C18 semipreparative column (Vydac, 218TP510, 10 mm×250 mm; 5 μm particle diameter; 300 Å pore size) equipped with a C18 guard column (Upchurch Scientific, AC-43 4.6 mm) at a flow rate of 3.5 mL/min. Further peptide purification was carried out by rechromatographing fractions on an analytical C18 column (Vydac, 238TP54, 4.6 mm×250 mm; 5 μm particle diameter; 300 Å pore size), with a flow rate of 1 mL/min. For semipreparative and analytical RP-HPLC separation, the buffers were 0.1% TFA (buffer A) and 0.1% TFA in 60% acetonitrile (buffer B). Peptides were eluted with an incremental linear gradient of 1% B/min. Absorbances were monitored at λ values of 220 and 280 nm. All HPLC fractions were manually collected, lyophilized, and kept at −40° C. prior to further use.

### Reduction and Alkylation of Cysteyl Residues.

Reduction and alkylation of cystine groups were carried out as previously described (Yen, T. Y., et al. (2002) J Mass Spectrom. 37, 15-30) with slight modifications. An aliquot of each peptide (˜1 pmol) was dried, re-dissolved in 0.1 M Tris-HCl (pH 6.2), 5 mM EDTA, 0.1% sodium azide and reduced with 6 mM DTT. Following incubation at 60° C., for 30 min, peptides were alkylated in a final volume of 15 μl with 20 mM IAM and 2 μl of NH₄OH (pH 10.5), at room temperature, for 1 h, in the dark. The reduced and alkylated peptides were purified using a Zip Tip (C18, size P10, Millipore).

### Peptide Sequencing.

Alkylated peptides were adsorbed onto Biobrene-treated glass fiber filters and amino acid sequences were carried by Edman degradation using an Applied Biosystems Procise model 491A Sequencer. The concentration of the peptides was determined by using the calibrated intensities of the first five PTH-amino acids residues on samples that were not reduced and alkylated.

### Molecular Mass Determination.

Positive ion MALDI-TOF mass spectrometry was carried out on an Applied Biosystems Voyager-DE STR spectrometer. Samples were dissolved in 0.1% TFA, 50% acetonitrile, and applied on α-cyano-4-hydroxycinnamic acid matrix. Spectra were obtained in the linear and reflector mode using Calmix 1 and Calmix 2 (Applied Biosystems) as external calibration standards.

### Disulfide Connectivity Analysis.

About 1 nmol of each lyophylized conopeptide was digested with a combination of CNBr and α-chymotrypsin prepared free of autolysis products and low molecular weight contaminants. In this case, peptides were not previously reduced and alkylated in order to maintain intact their disulfide bonds. Lysis of the peptides with CNBr (Acros Organics, USA) was performed using a mass ratio of 2:1 of CNBr to peptide (Simpson, R. J. (2002) Proteins and Proteomics: A Laboratory Manual.), in 20 μl of 70% formic acid. The peptidic sample was incubated for 20 h, at room temperature, in the dark, and the reaction was terminated by diluting the mixture with ˜5 volumes of H₂O following by removal of the excess free reagents by lyophylization with a Speed Vac concentrator. Subsequently, the sample was further digested with chymotrypsin (Sigma-Aldrich, USA), using a 1:20 ratio of chymotrypsin to substrate, in 20 μl of 0.1 M NH₄HCO₃ (pH 8.0), for 4 h, at 37° C. (Sudarslal, S. et al. (2003) FEBS Lett. 553, 209-212). Final digests were directly analyzed by MALDI-TOF mass spectrometry.

### Circular Dichroism Spectroscopy.

All circular dichroism (CD) measurements were carried out with a JASCO J-810 spectropolarimeter instrument using 200 μl solution of each peptide in HPLC water (final concentration of 3 μM; determined by normalized UV at λ=280 nm) in a quartz cell. Spectra were recorded over a λ=190-250 nm range at 25° C. using an average of 10 scans (scan speed of 100 nm/min). Peptide secondary structure was predicted using the method as previously described (Scholtz, J. M., et al. (1991) Parameters of helix-coil transition theory for alanine-based peptides of varying chain lengths in water Biopolymers 31, 1463-1470).

### NMR Spectroscopy.

NMR spectra were acquired on a Varian Inova 500 MHz instrument equipped with PFG, 3xRF channels and waveform generators. Nanomolar quantities of the native conopeptides directly isolated from the venom (vil14a=32 nmoles, flf14a=0.1 nmoles, flf 14b=3 nmoles, flf14c=0.6 nmoles) were dissolved in 40 μl of water with 10% D₂O (used for locking purposes) and 4 nanomoles of TSP and placed in 1.7 mm NMR tubes (Wilmad WG-1364-1.7). The pH was adjusted using 0.01 M solutions of HCl and NaOH and a Thermo micro-pH probe. Spectra were obtained using a Varian gHCN (generation 5) high performance 3 mm probe (pw90=3 μs, at the upper limit of the linear range of the RF amplifier) with a 1.7 mm capillary adaptor (Wilmad V-GFK-10/1.7). NMR experiments were recorded at pH 3.60 and at different temperatures (0, 10 and 25° C.) in order to achieve the best chemical shift dispersion possible to aid the sequence specific assignments. For 1D NMR experiments, the water signal was suppressed by using either WET (Smallcombe, S. H., et al. (1995), J. Mag. Res. 117, 295-303) or presaturation. In addition to the concentrations determined from sequencing, peptide concentrations were also evaluated by integrating the NMR signals of selected methyl groups and using the known concentration of TSP as an internal standard or the signal of selected methyl groups from peptides with known concentration as external standards. For 2D experiments, water suppression was carried out using WATERGATE (wg) (Piotto, M., et al. (1992) Gradient-tailored excitation for single-quantum NMR spectroscopy of aqueous solutions, J. Biomol. NMR 2, 661-665) in combination with 3919 purge pulses with flipback (Price, W. S. (1999) Water signal suppression in NMR spectroscopy, Annual reports on NMR spectroscopy, 38, 289-354), which were implemented in the TOCSY and NOESY pulse sequences. The wgTOCSY and wgNOESY experiments were used to obtain information on sequence-specific assignments and the secondary structure of the framework 14 conotoxins (Wuthrich, K. (1986) NMR of proteins and nucleic acids, Wiley & Sons, New York). All 2D-NMR spectra were recorded in the phase sensitive mode using the States-Haberkorn method (States, D. J., et al. (1982) J Magn. Reson. 48, 286-292) with a spectral width of 6000 Hz and 2K data points. For the wgTOCSY experiment, 160 scans for each of the 96 FIDs were acquired with relaxation delay of 1.7 s and a mixing time of 120 ms. The 2D wgNOESY spectra of the vi114 and flfl4 conopeptides were recorded using 256 scans for each of the 128 FIDs acquired with a 1.7 s relaxation delay and a mixing time of 200 ms. All 2D NMR data were processed using VNMR 6.1 C (Varian NMR Instruments) on Sun Blade 150 workstations. FIDs were apodized with a shifted sine bell window function and linearly predicted to 1K points in t1 and zero-filled to 2 k×2 k data matrices. The data was baseline corrected in F2 by applying a polynomial function. NOESY cross-peaks were assigned and classified according to their intensities as strong, medium or weak with the aid of their volume integrals. Sequence-specific assignments off all proton resonances were carried out using standard biomolecular NMR procedures (Wuthrich, K, supra).

Molecular Model of vil14a.

Molecular models were built by comparative modeling methods (Fiser, A. S., et al. (2003)Methods in Enzymol. 374, 463-493) based on the NMR structure of the κ-hefutoxin (PDB entry 1HP9) and the Om-toxins (PDB entries 1WQC-E) as templates and using the program Modeller (8v0). Briefly, conotoxin sequences were aligned according to the standard routine in the program using the PDB entries as templates (Table 1). A set of 20 model structures were built accordingly (John, B., and Sali, A. (2003) Comparative protein structure modeling by iterative alignment, model building and model assessment, Nucleic Acids Res. 31, 3982-3992). We selected the structure of better target Modeller energy. The final structure was evaluated for its agreement with the NOE NMR data. Molecular graphics were performed using UCSF Chimera beta vl (Pettersen, E. F., et al. (2004) UCSF Chimera—a visualization system for exploratory research and analysis, J. Comput. Chem. 25, 1605-1612).

### Nomenclature.

In this publication we adopted a nomenclature of three letters to designate Conus species, because the one or two letter nomenclature currently in place will not be enough to describe the large number of different non-fish-hunting species, especially those with similar first letter names. We decided to use the three letters “vil” to name the peptides from C. villepinii and “flf” for peptides from C. floridanus floridensis. Arabic numbers were used to represent the disulfide framework; since 13 has already been assigned to the a new 8-Cys/6-loop conotoxin, the framework described in this paper will become framework 14. The letter after the framework number indicates the order of elution on SE-HPLC.

### Example 1

### A Novel Conotoxin Framework with a Helix-Loop-Helix (Cs α/α) Fold

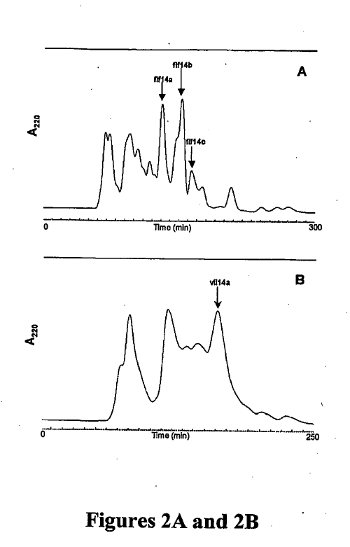

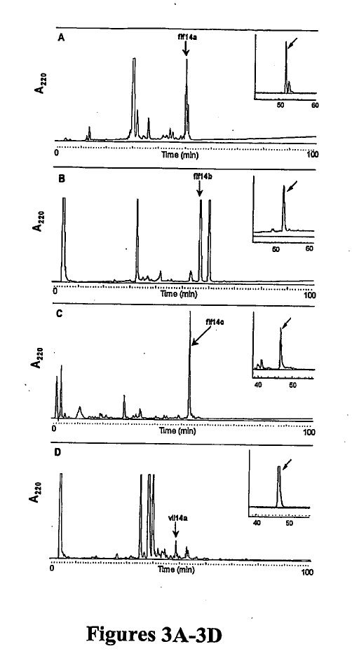

Peptide purification. Crude venom from C. villepinii (113 mg) and C. floridanus floridensis (97 mg) were initially fractionated using a SE-HPLC on the Superdex 30 column (FIGS. 2A and 2B). The flf14 peptides were separated by the Superdex-30 in spite of their similar size. This is not entirely unexpected, since this column is also known to partition analytes by hydrophobic interactions. The SE fractions identified by the arrows in FIGS. 2A and 2B were re-chromatographed on a semipreparative C18 column (FIGS. 3A-3D). Purification to single components was ultimately achieved using an analytical C18 column (FIGS. 3A-3D, inserts).

Reduction/Alkylation and peptide sequence determination. The purified peaks were subjected to reduction with DTT and alkylation with iodoacetamide. Mass spectrometry of the reduced/carboxymethylated peptides and of native peptides showed a mass difference consistent with the presence of four cysteine residues for each peptide. The reduced/alkylated conotoxins were sequenced to completion by Edman degradation. The sequences of the flf14 and vil14 conotoxins are shown in Table 1. Peptides flf14a and flf14c only differed by one amino acid residue. All of these conotoxins contained 27 residues and the four cysteines were separated by three loops of conserved size (loop 1=3 amino acids, loop 2=11 amino acids and loop 3=3 amino acids). This is a novel arrangement of Cys residues within conotoxin families that we have designated framework 14. Additionally, a BLAST search (Altschul, S. F., et al. (1997) Gapped blast and psi-blast: A new generation of protein database search programs, Nucleic Acids Res. 25, 3389-3402) of the databases (Swissprot/EMBL, PIR, PDB and nrdb95) did not show any significant sequence homology to reported proteins and peptides. Several 4-cystine/3-loop sequences (Table 1), including peptides from scorpion venom, κ-hefutoxins, κ-KTx1.3 and Om-toxins, were found to have similar loop spacing as the new conotoxins described here.

Mass spectrometry of purified peptides. Mass spectrometry carried out using MALDI-TOF in the reflector mode (M/ΔM resolution ˜10,000) yielded the following monoisotopic molecular ions: flf14a=3170.2 Da, flf14b=3098.0 Da, flf14c=3280.4 Da and vil14a=2872.5. Mass analysis of the reduced/carboxymethylated peptides and the native peptides showed a mass difference consistent with the presence of four cysteine residues in each peptide. The masses obtained for the peptides were in agreement with the calculated theoretical monoisotopic values determined for the assigned sequences and indicated that the peptides were not amidated at the C-terminus. The calculated molecular weights were obtained using Protein Prospector (Clauser, K. R., et al. (1999) Anal. Chem. 71, 2871-2882).

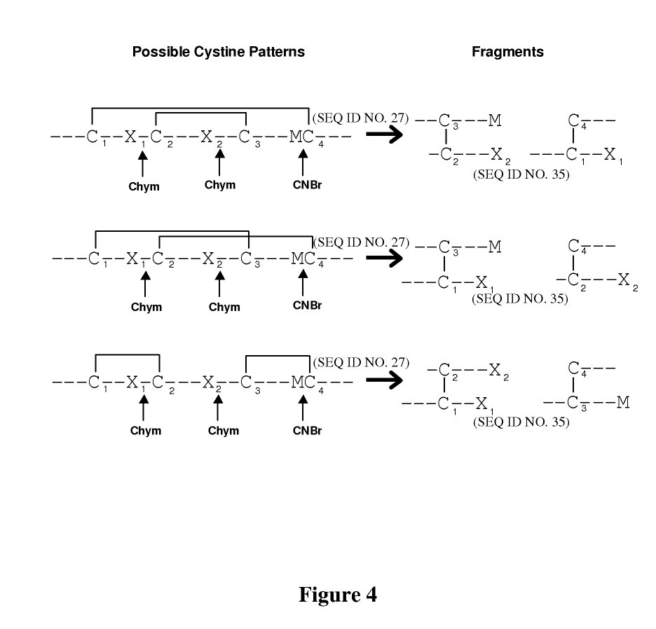

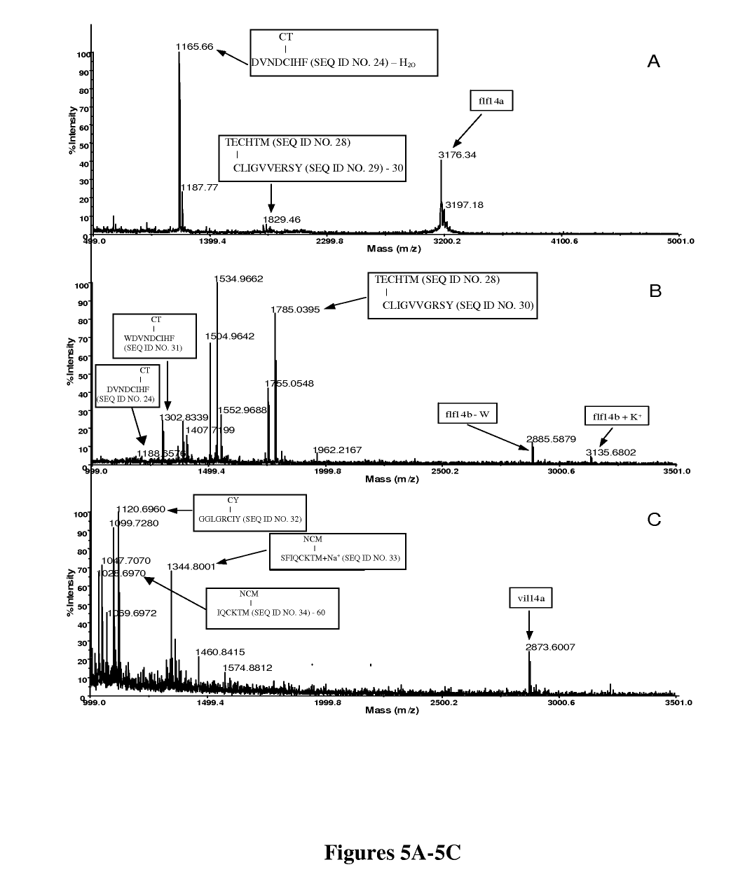

Disulfide connectivity analysis. The presence of four cysteine residues in these sequences suggested the existence of two disulfide bridges. Due to the limited quantities of native conotoxins available after their purification, the disulfide connectivity was established by cleaving the peptide bonds in between the loops of the native peptides (not reduced) in order to obtain fragments maintaining all disulfide bonds. The molecular weights of these fragments were determined by MALDI-TOF mass spectrometry, which only requires femtomole-quantities of sample. Three disulfide pairing patterns can be possible for these conotoxins and they can be distinguished using a combination of CNBr and chymotrypsin cleavage patterns (FIG. 4). Peptides flf14a, flf14b and vil14a contain a Met residue in the third loop which could be readily cleaved by CNBr. Further hydrolysis was then achieved by using chymotrypsin, which cleaves after aromatic residues Phe, Trp, Tyr. Disulfide pairing of flf14c was not determined as it lacks of Met residue in Loop3. MALDI-TOF mass spectra following treatment of each peptide with the combination of CNBr and chymotrypsin revealed that the disulfide pattern for these three peptides is C6-C26 and C10-C22 (FIGS. 5A-5C). No fragments representing other possible disulfide patterns were found. As expected, some fragments containing the Met showed a MH⁺-30 Da that corresponded to a homoserine lactone formed as a consequence of the CNBr treatment. Since vil14a contains two residues of Met, a loss of 60 Da was evidenced in the corresponding hydrolytic fragments of this peptide. A product of dehydration (MH⁺−18 Da) was obtained in the fragment DVNDCIHF bonded to CT. Dehydration has been reported for residues such as Ser, Asp, Glu and Thr. Other MS peaks that were observed represented intermediate products of digestion.

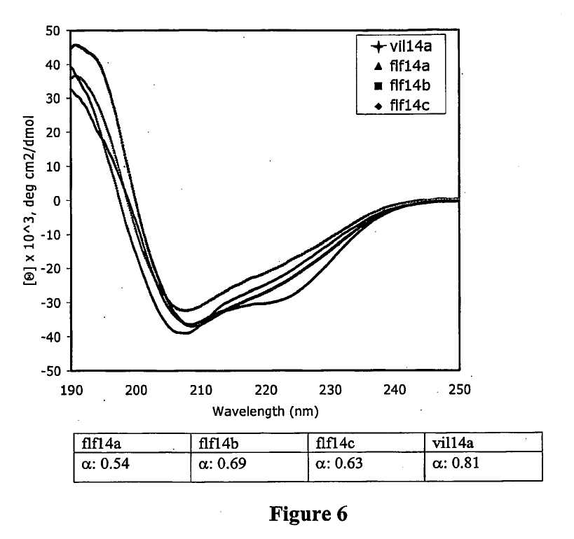

Circular dichroism spectra. CD spectra of flf14a, flf14b, flf14c and vil14a are shown overlaid in FIG. 6. Similar overall spectra were obtained for the four peptides with a maximum positive ellipticity at λ=195 nm and minimum with negative ellipticity at λ=208 nm. Conotoxin vil14a shows another minimum at λ=225 nm. Analyses performed using method proposed by Baldwin (Scholtz, J. M., Hong, Q., York, E. J., Stewart, J. M., and Baldwin, R. L. (1991) Biopolymers 31, 1463-1470) indicate that these peptides are predominantly α-helical, which is in good agreement with data derived from NMR spectroscopy.

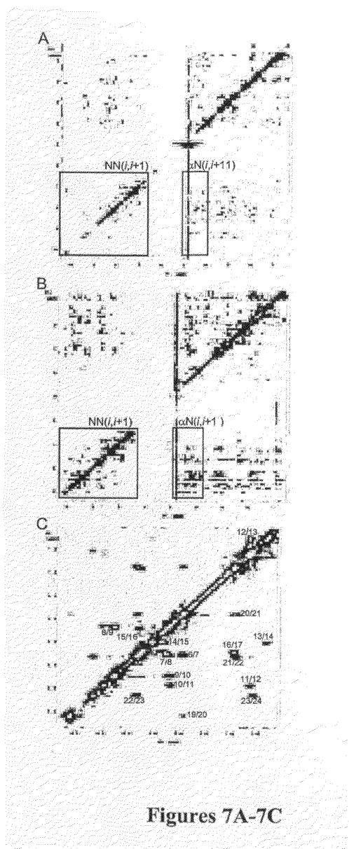

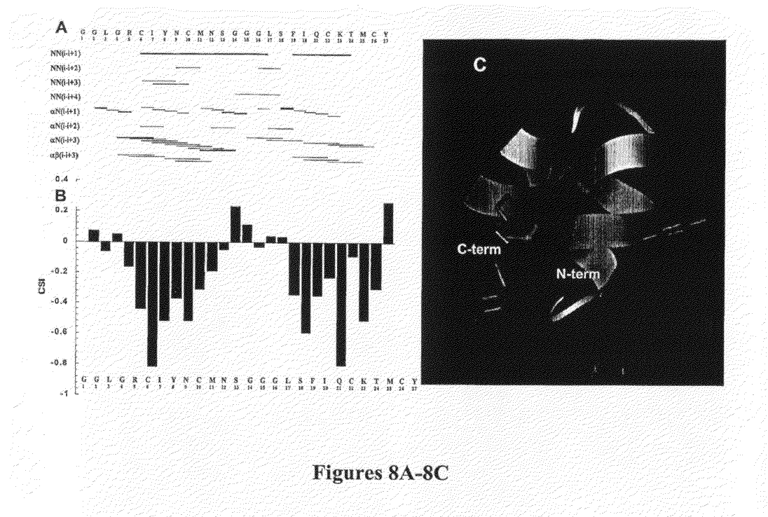

NMR Spectroscopy. We were able to obtain NMR spectra (1D and 2D) of nanomolar quantities (nanoNMR) of the flf14 and vil14 conotoxins directly isolated from the venom of the cone snails. In all cases, 2D wgTOCSY spectra were acquired; 2D wgNOESY with spectral quality suitable for structural determination (sufficient signal to noise ratio) were obtained for flf14b and vil14a. FIGS. 7A-7C shows wgNOESY spectra of flf14b (FIG. 7A) and vil14a (FIG. 7B) at 25° C. In spite of their small size (27 residues), a large number of NOE (>300) cross-correlations were found for these peptides. Spectra at lower temperatures (0° C. and 10° C.) resulted in even higher number of NOE cross-correlations. The flf14b and vil14a conotoxins have very well defined structure in solution at room temperature and in agreement with the results of CD results (FIG. 6). Sequence-specific assignments for all protons (except Gly1) in vil14a were achieved using the nanoNMR data (Table shown in FIG. 6). This allowed us to generate the NOE-based secondary structure connectivity map shown in FIG. 8A. 2D wgNOESY spectra revealed a high α-helical content for all four peptides. For vil14a, sequential NN(i, i+1) and αN(i, i+1) NOEs in conjunction with mid range NN(i, i+3), αN(i, i+3) and αβ(i, i+3) indicates two α-helical segments between residues 5-12 and 19-26 separated by the Gly-rich (with few NOEs) segment (13-18). The CSI plot of vil14a (FIG. 8B) indicated negative values for the same 5-12 and 19-26 segments, whereas the rest of amino acids showed positive or near zero values.

Molecular model of vil14a. A model of the 4-Cys/3-loop (1-4, 2-3 Cys pairing) with a helix-loop-helix fold found in vil14a (FIG. 8C) was built using the κ-hefutoxin (Srinivasan, K. N. et al. (2002) J. Biol. Chem. 277, 30040-30047) and Om-toxins (Chagot, B., et al (2005) Biochem. J. 388, 263-271) 3D structures as templates. Slight structural differences are found among these template structures; however, we selected the α-hefutoxin as the template for the model of vil14a shown in FIG. 8C, as the κ-hefutoxin-based model provided the lowest Modeller target energy.

Discussion: Here we detail the isolation of four novel Conus peptides from the venom of C. villepinii and C. floridanus floridensis that reveal a new conotoxin framework that highly differs from all known conotoxins. This new framework is characterized by a 4-Cys/3-loop sequence with a conserved loop spacing ¹Cys-X₃-²Cys-X₁₁-³Cys-X₃-⁴Cys and Cys pairing 1-4/2-3. We have termed this new arrangement framework 14. The only posttranslational modifications found in all these of 27-residue framework 14 conotoxins are cystine bridges. Unlike other 4-Cys conotoxins, vil14 and flf14 conotoxins are not amidated at the C-terminal end. While these peptides have the same framework, the inter-cystine amino acid sequences can be quite variable. Besides the cystines, the only conserved residue in all these four conotoxins is Ile7. flf14a differs from flf14b in residue 16 (E in flf14a vs G in flf13c); therefore, there are three distinct framework 14 conotoxins that show different degrees of sequence variability. The N-terminal tail and loop1 are homologous among the flf14 conotoxins. The central loop shows the greatest degree of variability among these three variants. Loop 3 is conserved between the flf14a/b and vill14a, but different for flf14c. This sequence variability is typical within conotoxin families, as interspecies sequence divergence, even in closely related Conus species (Woodward, S. R. et al. (1990) EMBO J. 9, 1015-1020) is usually observed. This new conotoxin framework was found in two different Conus species that exist in unrelated environments, as C. villepinii is restricted to deep-water habitats (>100 m), whereas C. floridanus is restricted to shallow water (<10 m). However, it has been recently suggested that C. floridanus and C. villepinii are in the same taxonomical clade (Duda, T. F., and Rolan, E. (2005) Molecular Ecology 14, 267-272).

It is likely that this new conotoxin framework defines a new conotoxin gene superfamily, as in most cases determined so far, new peptidic frameworks in Conus venom have defined new superfamilies. This is particularly true in this case, since the distribution of cysteine residues within the sequence is quite unique for Conus. Further work will compare the mRNA/cDNA sequences that encode for the precursor proteins of these conotoxins and evaluate if these precursors share a highly conserved and unique signal sequence that would define a gene superfamily to this new framework. However, this putative new superfamily would not be unique to Conus as other organisms, particularly scorpions, are capable of expressing this peptide framework.

Four other families of 4-Cys conotoxins have been described: α-conotoxins and ρ-conotoxins, which belong to the A-superfamily and ε-conotoxins and χ-conotoxins, which are members of the T-superfamily. α, ε and ρ-conotoxins have a ¹Cys-3Cys/²Cys-4Cys connectivity. By way of contrast, χ-conotoxins have ¹Cys-4Cys/²Cys-3Cys connectivity. We obtained a ¹Cys-4Cys/²Cys-3Cys pairing for the new framework 14 conotoxins (the same connectivity as χ-conotoxins) by digestion of three of these novel conotoxins with CNBr/chymotrypsin. Unlike all other 4-Cys conotoxins, the flf14 and vil14 conotoxins have all four cystine residues spaced out by loops (no vicinal Cys residues); this allowed the use of specific peptide bond cleavage in between the loops, thus releasing fragments that would reveal the Cys pairing. The advantage of this method is that it requires lower quantities of sample and it is less laborious, as the cleavage products can be directly detected by MALDI-TOF MS.

CD spectroscopy revealed a predominant α-helical content for all flf14 and vil14 conotoxins. This is an unusual structural feature, as most conotoxins are non-helical. Measurements of the molar ellipticity at λ=222 nm provided us with the relative helical contents of these framework 14 conotoxins. This approach yields good estimations of the relative helical content of these peptides. CD spectra for all flf14 were similar with one minima of ellipticity between λ=207-209 nm was observed. The CD spectrum of vil14a was slightly different with two distinct minima (λ=209 and 222 nm respectively). These slight differences in the CD spectra of the framework 14 conotoxins are consistent with slight structural differences in solution as observed in the three different NMR structures of Om-toxins.

Other 4-Cys/3-loop peptides with ¹Cys-4Cys/²Cys-3Cys pairing have been described. Notably, the κ-hefutoxins, κ-KTx1.3 and the Om-toxins, a family of K⁺ channel-binding scorpion toxins, share the same framework as these newly described conotoxins. However, prior to the discovery of this framework in the scorpion toxins, a sequence with similar characteristics has been described for MBP-1, an antibiotic peptide present in the seed of Zea mays (Duvick, J. et al. (1992) J. Biol. Chem. 267, 18814-18820) (Cys pairing was not determined for MBP-1). All of these peptides, including the flf14 and vil14 conotoxins, share this conserved framework where loops 1 and 3 invariable have three amino acids and loop 2 varies from 7 to 11 residues (Table 1). These peptides are highly α-helical according to their CD spectra and it has been determined by NMR that the κ-hefutoxin and the Om-toxins form a helix-loop-helix cystine-stabilized (Cs α/α) fold, where the two helices are linked by the two disulphide bridges.

NanoNMR analysis was used to address the details of the structural fold found in the flf14b and vil14a conotoxins. 3-32 nanomoles (9-90 μg) isolated directly from the cone snails were sufficient to yield spectra suitable for the structural analysis of these peptides. By way of contrast, 7 μmoles (19 mg) of synthetic peptide were utilized to determine the structure of the κ-hefutoxin. Similar synthetic quantities (˜1 μmole) were used for the structural determination of the Om-toxins. In spite of the severe resonance overlap, particularly of HN protons, sequence-specific assignments of all proton resonances were achieved for the vil14 conotoxin, allowing us to assess the structural features found in this new conotoxin fold. The 2D wgNOBSY of the flf14 and vil14 conotoxins at room temperature showed very well defined structures in solution, in a way that is reminiscent of the one observed in larger tightly folded globular proteins. The NMR structures of other two disulfide-bonded conotoxins (α, ε, ρ and χ) have been determined. However, these determinations have been carried out at lower temperatures and with a relatively low number of NOE constraints per residue. Highly structured frameworks have been observed in three disulfide-bonded conotoxins belonging to the O and M superfamilies, where a cystine knot with a triple-stranded β-sheet is the prevalent structural motif within these tightly compact scaffolds (Craik, D. J. et al. (2001), Toxicon 39, 43-60). This is not the case of the peptides flf14a, flf14b, flf14c and vil14a, since here we have two disulfide bonds and all four cystine residues are spaced by loops of at least three amino acids. Instead, two α-helical distinct segments, 5-12 and 19-26, are clearly identified by their NOE connectivities and their chemical shift indexes. This is in good agreement with the results of CD spectra, which revealed a predominantly α-helical secondary structure for these four new conotoxins. Most conotoxin frameworks described to date are non helical, with the exception of the α and ρ conotoxins, which have a short helical segment, and some M-Superfamily conotoxins.

The flf14 and vil14 conotoxins have the same helix-loop-helix Cs α/α fold first described for the κ-hefutoxins from the scorpion Heterometrus fulvipes. The κ-hefutoxins blocks the voltage-gated K⁺ channels, Kv1.2 and Kv1.3 and also slows the activation kinetics of the Kv1.3 currents. Other related scorpion toxins, the κ-KTx 1.3 from Heterometrus spinifer and the Om-toxins from the Opisthacanthus madagascarienses share this same fold and they show differential targeting of the K⁺ channels. In addition to the Cys residues, the κ-hefutoxins have several amino acids in common with the vil14 and flf14 conotoxins. Examination of existing 4-Cys frameworks with a ¹Cys-4Cys/²Cys-3Cys pairing reveals several related non-helical toxins such as the χ-conotoxins from Conus marmoreus, which target the noradrenaline transporter; Tachyplesin, a peptide with antimicrobial properties isolated from the horseshoe crab Tachypleus tridentatus and Gomesin, also an anti-microbial peptide from hemocytes of the spider Acanthoscurria gomesiana. However, their 3D fold is quite different from the framework 14 conotoxins.

Comparison of the loop size of the vil14 and flf14 conotoxins with Cs α/αscorpion toxins shows that the first and third loops have the same 3-amino acid spacing (Table 1); the second loop of κ-hefutoxins is two residues shorter. Even shorter spacing of the central loops can be found in the Tachyplesin and Gomesin (3/5/3); however, these peptides are not helical and they exhibit a Cs β/β fold. Therefore, the length of the central loop is critical in determining the overall fold of these motifs.

Structural analysis of our nanoNMR data confirmed that the flf14 and vil14 conotoxins have the same three-dimensional fold as the κ-hefutoxins and Om-toxins. It is quite remarkable that we found a new conotoxin framework in two Conus worm-hunting species that resemble so closely these newly discovered Cs α/α K⁺ channel-binding scorpion toxins. Just as other potassium channel toxins, the vil14 conotoxin has a functional Lys/Tyr (or Phe) dyad separated by 6.0±1.0 Å. This is consistent with the model built for the vil14a conotoxin, based on the NMR structure of the κ-hefutoxin. This model is in agreement with our current nanoNMR data and only small variations with nanoNMR-based 3D structure of the vil14a. Furthermore, the Cs α/α scorpion toxins only show small structural variations among themselves. The location of Lys23 (in blue in FIG. 8C) indicates two proximal Tyr residues (Y8 and Y27). Therefore, two potential dyad pairs can be considered for the K⁺ channel-binding properties of vil14a. This dyad was found in structurally unrelated potassium channel-blocking toxins from scorpions, sea anemones and in the κ-conotoxins. The vil14a conotoxin has a putative functional dyad in residues Tyr8/Lys23 or Tyr27/Lys23. Given that the κ-hefutoxins and Om-toxins bear the dyad and clearly are structurally related to the framework 14 conotoxins, we can extend the dyad binding mode to the new framework 14 conotoxins. We have carried out experiments that indicate that vil14a indeed blocks K⁺ channels in PC12 cells. However, the sequences of flf14 conotoxins do not have Lys residues and no apparent dyad is contained in these conotoxins. This is also the case with OmTx3, the only Om-toxin (Table 1) that lacks the functional dyad; however, it is the best blocker of the Om-toxin set. It has been proposed that other residues positively charged may mimic K⁺ ions entering the pore, occluding the ion pathway. Alternate modes of binding to the K⁺ channels, may exclude the aromatic amino acid as critical for binding, or that completely exclude the functional dyad, have been suggested. The binding determinants and the differential targeting of these conotoxins are currently being addressed with the appropriate electrophysiological experiments.

The commonalities of these new framework 14 conotoxins with the recently described Cs α/α K⁺ channel binding toxins found in scorpions are an indication of a shared molecular imprint in these unrelated predatory venomous animals. While the venom of these animals is a rich source of Cys-constrained neuroactive peptides, the detailed frameworks, number of Cys residues, the arrangement of Cys with the sequence, loop sizes, Cys pairing and length of the polypeptide chains, generally differs. Scorpion toxins are larger, with more Cys bridges and their frameworks are different from the ones found in conotoxin families and superfamilies. In fact, the gene arrangements are different as toxin precursors are encoded by three exons in cone snails as opposed to two exons in scorpions. However, all of these toxins are probably related by common ancestral genes which were initially found in scorpions. Ancient scorpions of aquatic origin are estimated to appear 400 Mya, whereas cone snails date to 55 Mya according to the fossil record. While Conus venom toxins are considered molecularly more diverse, as they are capable of a combinatorial plethora of posttranslational modifications and express linear families of conopeptides, it is unclear how much commonality there is between Conus peptides and peptides found in other organisms since only a small fraction of either the Conus peptide library or the scorpion library toxins library has been characterized. Another conotoxin framework, specifically the classical O-superfamily conotoxin C—C—CC—C—C array, has been found in spiders, sea sponges and within the viral genomes. However, in the case of the fll14 and vil14 conotoxins, cone snails are utilizing the same biochemical strategy as scorpions in targeting ion channels through this particular Cs α/α framework in order to capture their meal. Further structural and functional studies of these conotoxins will provide a better understanding into the usefulness of this new scaffold in molecular engineering and neuronal targeting and could lead to the design of neuropharmacological agents that target specific receptors or ion channels.

### Example 2

### The F14 Conotoxins Define a New Gene Superfamily

Upon extraction of the mRNA from the venom duct of C. villepinii, the mRNA was used as a template to produce its corresponding cDNA. The cDNA was amplified by PCR using part of vil14a sequence as a primer. The resulting cDNA was cloned into competent cells and the selected DNA was extracted and submitted to DNA sequencing. The detailed procedure is shown below.

Procedure: The venom duct was removed from the fresh killed animal and stored at −70° C. Approximately, 1.5 cm of the duct was taken for the RNA isolation (RNeasy Mini Kit, Qiagen). 5′ RACE was performed using first strand cDNA transcripted with the smart race system (Clontech). The 5′ RACE reverse primer (5′-TARCACATNGTYTTRCAYTGDATRAA-3′) (SEQ ID NO: 21) was based on the peptide sequence FIQCKTMCY (SEQ ID NO: 22). The 5′ RACE products were ligated into the T-tailed plasmid vector pGEM-Teasy (Promega) and transformed into competent cells (JM 109, Promega). Positive clones containing inserts of the expected size were picked and isolated using the QIAprep Spin Miniprep kit (Quiagen) and sequenced.

F-Superfamily: vil14a precursor

MRRPEVRRPEVRQPEFAETPVGQKRGGLGRCIYNCMNSGGGLSFIQCKT MC (SEQ ID NO: 20). The vil14a precursor show a signal region, whose sequence has no homology to any other conotoxin superfamily, thus defining a novel gene superfamily that we have designated as the F-superfamily.

Vil14a blocks Kv1.3 potasium channels: The flf14 and vil14 conotoxins have the same helix-loop-helix Cs α/αfold first described for the κ-hefutoxins from the scorpion Heterometrus fulvipes. The κ-hefutoxins blocks the voltage-gated K⁺ channels, Kv1.2 and Kv1.3 and also slows the activation kinetics of the Kv1.3 currents. Other related scorpion toxins, the κ-KTx1.3 from Heterometrus spinifer and the Om-toxins from the Opisthacanthus madagascarienses, share this same fold and they show differential targeting of the K⁺ channels. In addition to the Cys residues, the κ-hefutoxins have several amino acids in common with the vil14 and flf14 conotoxins. Structural analysis of our nanoNMR data confirmed that the flf14 and vil14 conotoxins have the same three-dimensional fold as the κ-hefutoxins and Om-toxins. It is quite remarkable that we found a new conotoxin framework in two Conus worm-hunting species that resemble so closely these newly discovered Cs α/α K⁺ channel-binding scorpion toxins. Just as other potassium channel toxins, the vil14 conotoxin has a functional Lys/Tyr (or Phe) dyad separated by 6.0±1.0 Å. This is consistent with the model built for the vil14a conotoxin, based on the NMR structure of the κ-hefutoxin. This model is in agreement with our current nanoNMR data and only small variations with nanoNMR-based 3D structure of the vil14a are expected. Furthermore, the Cs α/α scorpion toxins only show small structural variations among-themselves. The location of Lys23 (FIG. 8C) indicates two proximal Tyr residues (Y8 and Y27). Therefore, two potential dyad pairs can be considered for the K⁺ channel-binding properties of vil14a. This dyad was found in structurally unrelated potassium channel-blocking toxins from scorpions, sea anemones and in the ic-conotoxins. The vil14a conotoxin has a putative functional dyad in residues Tyr8/Lys23 or Tyr27/Lys23. G

Our experiments indicate that vil14a blocks K⁺ channels in PC12 cells. However, the sequences of flf14 conotoxins do not have Lys residues and no apparent dyad is contained in these conotoxins. This is also the case with OmTx3, the only Om-toxin (Table 1) that lacks the functional dyad; however, it is the best blocker of the Om-toxin set. Other residues positively charged may mimic K⁺ ions entering the pore, occluding the ion pathway. The binding determinants and the differential targeting of these conotoxins were addressed with the appropriate electrophysiological experiments described below. vil14a binds the Kv1.3 potassium channels transfected intl HEK293 cells with a Kd=15.6 μM.

Transient transfection: Human embryonic kidney (EK293) cells were grown to ˜70% confluence in DMEM-F12 (Life Technologies, Carlsbad Calif.) containing 10% fetal bovine serum, 1% penicillin and streptomycin solution (Life Technologies) at 37° C., in 5% CO₂ one day before transfection. Then transfected with 8-10 μg of Kv1.3 plasmid and 1-2 μg reporter plasmid CD8 using Lipofectamine 2000 (Invitrogen, Carlsbad, Calif.) according to the manufacturer's instructions. Cells were replated over poly-L-Lysine covered coverslips. 15 h after transfection. Transfection-positive cells were identified by immunobeads (CD8-Dynabeads M-450, Brown Deer, Wis.). Electrophysiological studies were performed 16-48 h post transfection.

To identify cells with a high probability of expressing recombinant transporters, cells were cotransfected with a plasmid encoding the CD8 antigen and incubated 5 min before use with polystyrene microbeads precoated with anti-CD8 antibodies (Dynabeads M-450 CD8, Dynal, Great Neck, N.Y.). Only cells decorated with microbeads were used for electrophysiological recordings.

Whole-cell voltage clamp: Whole-cell configuration was used to record K⁺ currents in cells coated with CD-8 beads. Pipettes electrodes were pilled from thin-walled borosilicate glass (Sutter Instruments). Electrodes had a resistance of 1-2 MΩ. Whole-cell recordings were made according to standard techniques with a commercially available patch-clamp amplifier (Axopatch 200-A, Axon Instruments, Foster City, Calif., USA) in an Axiovert 10 inverted microscope (Zeiss, Jena, Germany). Data were acquired, using pClamp 9 software (Axon Instruments; Foster City, Calif., U.S.A.). Capacitive and leak currents have been subtracted from the data shown. Capacitive transients were cancelled with the electronic circuitry provided with the amplifier, whereas leak currents were cancelled by pClamp leak substraction protocol (P/−6). Cells were held at −90 mV. The external solution for whole cell K⁺ currents contained: (in mM) 150 NaCl, 1 MgCl₂, 4 KCl, 2 CaCl₂, 10 HEPES, (pH 7.4 with NaOH). Pipette electrodes contained (in mM) 144 KCl, 1 EGTA and 10 HEPES (pH 7.2 with KOH). All experiments were performed at room temperature (21-22° C.).

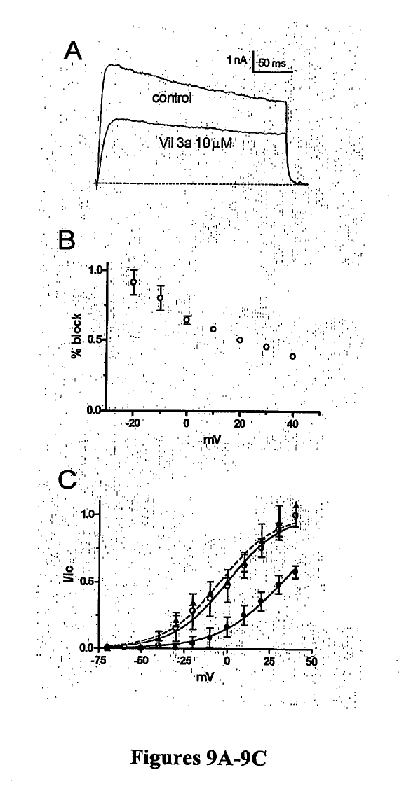

Functional characterization of vil14a: The functional effect of vil14a was investigated in HEK293 expressing Kv1.3. Vil14a (10 μM) reduced currents by ≈39%. The dose dependent reduction in the K⁺ current was voltage dependent as shown in figure xB, the degree of block increased with the test potentials from −20 to +40 mV. Block by Vil14a is reversible as shown in FIG. 9.

TABLE 1

| Peptide | Sequence | Source |
|---|---|---|
| flf14a | 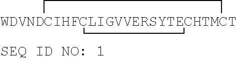 | Conus floridanus floridensis |
| flf14b | 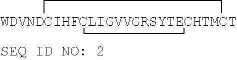 | Conus floridanus floridensis |
| flf14c | WDAYDCIQFCMRPEMRHTYAQCLSICT | Conus floridanus floridensis |
| | SEQ ID NO: 3 | |
| vil14a | 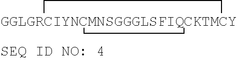 | Conus villepinii |
| κ-Hefutoxin 1 | G--HACYRNCWRE-GNDEETCKERC | Heterometrus fulvipes (Scorpion) |
| | SEQ ID NO: 5 | |
| κ-Hefutoxin 2 | G--HACYRNCWRE-GNDEETCKERCG | Heterometrus fulvipes (Scorpion) |
| | SEQ ID NO: 6 | |
| κ-KTx1.3 | G--F-CYRSCWKA-GHDEETCKKECS | Heterometrus spinifer (Scorpion) |
| | SEQ ID NO: 7 | |
| OmTX1 | ---DPCYEVCLQQHGNV-KECEEACKHPVE | Opisthacanthus madagascarienses |
| | SEQ ID NO: 8 | (Scorpion) |
| OmTx2 | ---DPCYEVCLQQHGNV-KECEEACKHPVEY | Opisthacanthus madagascarienses |
| | SEQ ID NO: 9 | (Scorpion) |
| OmTx3 | N--DPCYEVCLQHTGNV-KACEEACQ | Opisthacanthus madagascarienses |
| | SEQ ID NO: 10 | (Scorpion) |
| OmTx4 | ---DPCYEVCLQQHGNV-KECEEACKHP | Opisthacanthus madagascarienses |
| | SEQ ID NO: 11 | (Scorpion) |
| MBP-1 | RSGRGECRRQCLRRHEGQPWETQECMRRCRR | Zea mays (Corn) |
| | RG SEQ ID NO: 12 | |
| Tachyplesin | ---KWCFRVCYRGI-------CYRRCR | Tachypleus tridentatus (Horshoe crab) |
| | SEQ ID NO: 13 | |
| Gomesin | ----ZCRRLCYKQR-------CNTYCRGR | Acanthoscurria gomesiana (Spider) |
| | SEQ ID NO: 14 | |

TABLE 2
Amino acid sequences of peptides: TES, PUR-Mdb,
PUR-Mdb, ERM-Mde, ERM-Mhe

| | | |
|---|---|---|
| Conophan | | |
| TES_B05d050607b | VYHAHPYSNAVWS | SEQ ID NO: 15 |

α-Conotoxins - Target: nicotinic acetyicholine
receptor (Skeletal muscle subtype)

| | | |
|---|---|---|
| PUR_Mdb | -SGCCKHPACGKNRC | SEQ ID NO: 16 |
| PUR_Mda | -SGCCKHOACGKNRC | SEQ ID NO: 17 |
| ERM_Mde | GOGCCWNPACVKNRCR | SEQ ID NO: 18 |
| ERN_Mhe | RDPCCSNPACNVNNPQIC* | SEQ ID NO: 19 |

V = D-Valine
O = γ-hydxoxyproline

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

All references cited herein, are incorporated herein by reference.

What is claimed is:

1. An isolated conopeptide, comprising a framework characterized by a 4-Cys/3-loop sequence with a conserved loop spacing ¹Cys-X₃-²Cys-X₁₁-³Cys-X₃-⁴Cys and Cys pairing 1-4/2-3.

2. The isolated conopeptide of claim 1, wherein the conopeptide comprises a Lys/Tyr diad.

3. The isolated conopeptide of claim 1, wherein the peptide is at least one of SEQ ID NO's: 1-4 and 14-20

4. A composition comprising an isolated conopeptide, wherein the isolated conopeptide comprises a framework characterized by a 4-Cys/3-loop sequence with a conserved loop spacing ¹Cys-X₃-²Cys-X₁₁-³Cys-X₃-⁴Cys and Cys pairing 1-4/2-3 in a pharmaceutically acceptable carrier.

5. The composition of claim 4, wherein the conopeptide comprises a Lys/Tyr diad.

6. The composition of claim 4, wherein the conopeptide is at least one of SEQ ID NO's: 1-4 and 14-20

7. An isolated conopeptide comprising at least one of SEQ ID NO's.: 1-4 and 14-20.

8. A composition comprising at least one of SEQ ID NO's.: 1-4 and 14-20 in a pharmaceutical carrier.

9. A method for inducing analgesia in a mammal comprising administering a therapeutically effective amount of a conopeptide identified by an one of SEQ ID NO's.: 1-4 and 14-20.

10. The method of claim 9, wherein said administration comprises using a delivery means selected from the group consisting of a pump, microencapsulation, a continuous release polymer implant, microencapsulation, naked or unencapsulated cell grafts, injection and oral administration.

11. The method of claim 9, wherein the amount of conopeptide administered is between about 0.001 mg/kg to about 250 mg/kg.

12. A pharmaceutical composition comprising a therapeutically effective amount of a conopeptide identified by at least one of SEQ ID NO's.: 14 and 14-20 or a pharmaceutically acceptable salt or solvate thereof and a pharmaceutically acceptable carrier.

13. The composition of claim 12, which further comprises one or more drugs useful in the treatment of pain.

14. A method of treating cancer comprising: administering a therapeutically effective amount of a conopeptide identified by an one of SEQ ID NO's.: 1-4 and 14-20 to a patient in need thereof, and, treating cancer.

15. An isolated cell comprising a vector expressing a conopeptide, comprising a framework characterized by a 4-Cys/3-loop sequence with a conserved loop spacing ¹Cys-X₃-²Cys-X₁₁-³Cys-X₃-⁴Cys and Cys pairing 1-4/2-3.

16. The isolated cell of claim 15, wherein the expressed conopeptide comprises a Lys/Tyr diad.

17. The isolated cell of claim 15, wherein the expressed peptide is at least one of SEQ ID NO's: 1-4 and 14-20.

18. A method of blocking potassium channels in a cell comprising:
transforming a cell with a vector expressing a conopeptide, comprising a framework characterized by a 4Cys/3-loop sequence with a conserved loop spacing ¹Cys-X₃-²Cys-X₁₁-³Cys-X₃-⁴Cys and Cys pairing 1-4/2-3; and,
culturing said cell, wherein,
expression of the conopeptide blocks the potassium channel.

19. The method of claim 18, wherein the expressed conopeptide comprises a Lys/Tyr diad.

20. The method of claim 18, wherein the expressed conopeptide is at least one of SEQ ID NO's: 1-4 and 14-20.


SEQUENCE LISTING (36 sequences)

SEQ ID NO: 1 - protein, 27 aa, Conus floridanus floridensis
WDVNDCIHFCLIGVVERSYTECHTMCT

SEQ ID NO: 2 - protein, 27 aa, Conus floridanus floridensis
WDVNDCIHFCLIGVVGRSYTECHTMCT

SEQ ID NO: 3 - protein, 27 aa, Conus floridanus floridensis
WDAYDCIQFCMRPEMRHTYAQCLSICT

SEQ ID NO: 4 - protein, 27 aa, Conus villepinii
GGLGRCIYNCMNSGGGLSFIQCKTMCY

SEQ ID NO: 5 - protein, 22 aa, Heterometrus fulvipes
GHACYRNCWREGNDEETCKERC

SEQ ID NO: 6 - protein, 23 aa, Heterometrus fulvipes
GHACYRNCWREGNDEETCKERCG

SEQ ID NO: 7 - protein, 22 aa, Heterometrus spinifer
GFCYRSCWKAGHDEETCKKECS

SEQ ID NO: 8 - protein, 26 aa, Opisthacanthus madagascarienses
DPCYEVCLQQHGNVKECEEACKHPVE

SEQ ID NO: 9 - protein, 27 aa, Opisthacanthus madagascarienses
DPCYEVCLQQHGNVKECEEACKHPVEY

SEQ ID NO: 10 - protein, 23 aa, Opisthacanthus madagascarienses
NDPCYEVCLQHTGNVKACEEACQ

SEQ ID NO: 11 - protein, 24 aa, Opisthacanthus madagascarienses
DPCYEVCLQQHGNVKECEEACKHP

SEQ ID NO: 12 - protein, 33 aa, Zea mays
RSGRGECRRQCLRRHEGQPWETQECMRRCRRRG

SEQ ID NO: 13 - protein, 17 aa, Tachypleus tridentatus
KWCFRVCYRGICYRRCR

SEQ ID NO: 14 - protein, 18 aa, Acanthoscurria gomesiana
ZCRRLCYKQRCVTYCRGR

SEQ ID NO: 15 - protein, 13 aa, Conus sp.
VYHAHPYSNAVWS

SEQ ID NO: 16 - protein, 14 aa, Conus sp.
SGCCKHPACGKNRC

SEQ ID NO: 17 - protein, 14 aa, Conus sp.
  gamma hydroxyproline (7)..(7)
SGCCKHPACGKNRC

SEQ ID NO: 18 - protein, 16 aa, Conus sp.
  gamma hydroxyproline (2)..(2)
GPGCCWNPACVKNRCR

SEQ ID NO: 19 - protein, 18 aa, Conus sp.
RDPCCSNPACNVNNPQIC

SEQ ID NO: 20 - protein, 51 aa, Conus villepinii
MRRPEVRRPEVRQPEFAETPVGQKRGGLGRCIYNCMNSGGGLSFIQCKTMC

SEQ ID NO: 21 - protein, 26 aa, ARTIFICIAL
  primer
TARCACATNGTYTTRCAYTGDATRAA

SEQ ID NO: 22 - protein, 9 aa, Conus villepinii
FIQCKTMCY

SEQ ID NO: 23 - protein, 7 aa, Conus sp.
  misc_feature (2)..(2): Xaa can be any naturally occurring amino acid
CXCXCXC

SEQ ID NO: 24 - protein, 8 aa, Conus sp.
DVNDCIHF

SEQ ID NO: 25 - protein, 4 aa, Conus sp.
CCCC

SEQ ID NO: 26 - protein, 6 aa, Conus sp.
CCCCCC

SEQ ID NO: 27 - protein, 7 aa, Conus sp.
  misc_feature (2)..(2): Xaa can be any naturally occurring amino acid
CXCXCMC

SEQ ID NO: 28 - protein, 6 aa, Conus sp.
TECHTM

SEQ ID NO: 29 - protein, 10 aa, Conus sp.
CLIGVVERSY

SEQ ID NO: 30 - protein, 10 aa, Conus sp.
CLIGVVGRSY

SEQ ID NO: 31 - protein, 9 aa, Conus sp.
WDVNDCIHF

SEQ ID NO: 32 - protein, 8 aa, Conus sp.
GGLGRCIY

SEQ ID NO: 33 - protein, 8 aa, Conus sp.
SFIQCKTM

SEQ ID NO: 34 - protein, 6 aa, Conus sp.
IQCKTM

SEQ ID NO: 35 - protein, 4 aa, Conus sp.
  misc_feature (4)..(4): Xaa can be any naturally occurring amino acid
MCCX

SEQ ID NO: 36 - protein, 4 aa, Conus sp.
  misc_feature (1)..(1): Xaa can be any naturally occurring amino acid
XCCX